(19) United States
(12) Patent Application Publication
(10) Pub. No.: US 2018/0085331 A1
(43) Pub. Date: Mar. 29, 2018

(54) COMPOSITIONS FOR MITIGATING HEAD TRAUMA

(71) Applicant: Garrett E. Wdowin, Corona del Mar, CA (US)

(72) Inventor: Garrett E. Wdowin, Corona del Mar, CA (US)

(21) Appl. No.: 15/276,554

(22) Filed: Sep. 26, 2016

(51) Int. Cl. A61K 31/202; A61K 9/06; A61K 31/12; A61K 31/05; A61K 31/201; A61K 45/06; A61K 9/00

(52) U.S. Cl. CPC A61K 31/202; A61K 9/06; A61K 31/12; A61K 9/0053; A61K 31/201; A61K 45/06; A61K 31/05

(57) ABSTRACT

Compositions and methods and provided for preventing or mitigating the effects of traumatic brain injuries, such as concussions. The compositions comprises between 100-11500 mg Docosahexaenoic acid (DHA), 100-3000 mg curcumin, and 100-3000 mg of resveratrol. The compositions may further include 100-3000 mg of α-linoleic acid. In some embodiments, the compositions may be administered to a subject prior to, during, or after a period of time wherein a traumatic brain injury may occur or may have occurred.

### FIELD OF THE INVENTION

The field of the present disclosure generally relates to treating and preventing the neurological damage associated with head trauma injuries. More particularly, the field of the invention relates to compositions and methods for preventing and mitigating head injuries and physical trauma to the brain.

### BACKGROUND

A traumatic brain injury occurs when an external mechanical force causes damage to the brain. Traumatic brain injuries are classified based on severity, the anatomical features of the injury, and the mechanism of the injury. Concussions are one of the most common forms of traumatic brain injury. A concussion can be defined as a complex pathophysiological process affecting the brain, induced by traumatic biomechanical forces.

Several common features that incorporate clinical, pathological and biomechanical injury constructs that may be utilized in defining the nature of a concussive head injury include: (1) concussion caused either by a direct blow to the head, face or neck or a blow elsewhere on the body with an “impulsive” force transmitted to the head; (2) concussion typically resulting in a rapid onset of short-lived impairment of neurologic function that resolves spontaneously; (3) concussion resulting in neuropathological changes wherein acute clinical symptoms largely reflect a functional disturbance rather than a structural injury; (4) concussion resulting in a graded set of clinical symptoms that may involve a loss of consciousness; and (5) concussion wherein no abnormality is observable on standard structural neuroimaging studies. Resolution of the clinical and cognitive symptoms typically follows a sequential course. In a small percentage of cases, however, post-concussive symptoms may be prolonged and even lead to other neurological diseases, including chronic traumatic encephalopathy (CTE).

Concussions may result in a variety of symptoms including headache, physical impairment, such as unsteadiness, cognitive impairment, such as, for example, confusion or memory loss, as well as abnormal behavior. Traumatic brain injuries such as concussions are complex and involve a constellation of signs and symptoms that vary from patient to patient.

Traditionally, concussions have been treated through physical and cognitive rest after the event has taken place. Few preventative measures have been taken outside of improved protective equipment, such as helmets, neck braces, protective pads, and the like. This is due in part to a perceived spontaneity of traumatic events. It has been estimated that as many as 3.8 million sports and recreation-related concussions occur in the United States each year. Over 60% of these concussions occur during a period wherein blows to the head were expected to occur, such as during a contact sport.

What is needed, therefore, is a composition that may be taken prior to a period of time wherein a traumatic brain injury may be sustained, or following an occurrence of such an injury. It is contemplated that such a composition may advantageously include a variety of natural and essential ingredients that provide nutritional building blocks and powerful antioxidants for the brain so as to lessen acute damage associated with head trauma and to assist in neurological repair following a head trauma.

### SUMMARY OF THE INVENTION

Compositions and methods are described for preventing or mitigating the effects of head injuries or brain trauma, such as concussions, and accelerating the healing thereof. Accordingly, in one exemplary embodiment, a composition is provided comprising components selected from a group including Docosahexaenoic acid (DHA), curcumin and resveratrol. An advantage of this composition is that it serves several functions, including ingredient synergy, limiting inflammation, improving energy production (e.g., supporting mitochondrial function by increasing ATP production and enhancing energy cellular processes such as the Electron Transport Chain), augmenting circulation, stimulating neurogenesis, upregulating the production of new neurons in adult brains, enhancing neural plasticity and repair, restoring capillary integrity, acting as a neuro-protective and cytoprotective agent by limiting oxidative stress, and promoting repair of damaged cervical musculature and connective tissue. As such, the composition is useful for reducing the duration and/or severity of concussive symptoms and associated neurological damage from head trauma.

In an exemplary embodiment, the composition comprises between 100 mg and 11500 mg of DHA, between 100 mg and 3000 mg of curcumin, and between 100 mg and 3000 mg of resveratrol. In another exemplary embodiment, the composition comprises an oral composition that may be easily absorbed. In another exemplary embodiment, the oral composition is comprised of a solid, semisolid, gel, slurry, paste, liquid, crystalline or encapsulated form. In another exemplary embodiment, the oral composition may be implemented in the form of a capsule or a tablet. In yet another exemplary embodiment, the composition further comprises at least one component selected from a group comprised of linoleic acid (n-6), or α-linoleic acid (n-3). In another exemplary embodiment the composition includes trans-resveratrol, cis-resveratrol, or a combination thereof.

In an exemplary embodiment, a method is provided for preventing traumatic brain injury, for example a concussion, wherein the method comprises administering an effective amount of a composition comprising at least a portion of DHA ranging between 100 mg and 11500 mg, a portion of curcumin ranging between 100 mg and 3000 mg, and a portion of resveratrol ranging between 100 mg and 3000 mg.

In an exemplary embodiment, a composition for mitigating head trauma may be administered to a subject one hour prior to a period wherein a traumatic head injury may be sustained. In another exemplary embodiment, the composition may be administered between one hour and twenty-four hours, inclusive, prior to the period wherein a traumatic head injury may be sustained. In another exemplary embodiment, the composition may be administered to the subject during the period wherein a traumatic head injury may be sustained. In another exemplary embodiment, the composition may be administered to the subject up to one hour after a traumatic head injury has been sustained. In another exemplary embodiment, the composition may be administered between one hour and twenty-four hours, inclusive, after the period wherein a traumatic head injury has been sustained. In another exemplary embodiment, the composition may be administered before, during and after the period wherein a traumatic head injury may be sustained.

### DETAILED DESCRIPTION

In the following description, numerous specific details are set forth in order to provide a thorough understanding of the present disclosure. It will be apparent, however, to one of ordinary skill in the art that the invention disclosed herein may be practiced without these specific details. In other instances, specific numeric references such as “a first compound,” may be made. However, the specific numeric reference should not be interpreted as a literal sequential order but rather interpreted that the “first compound” is different than a “second compound.” Thus, the specific details set forth are merely exemplary. The specific details may be varied from and still be contemplated to be within the spirit and scope of the present disclosure. Further, as used herein, the terms “about,” “approximately,” or “substantially” for any numerical values or ranges indicate a suitable dimensional tolerance that allows the part or collection of components to function for its intended purpose as described herein.

As used herein, the terms “composition” and “pharmaceutical composition” are to be construed as equivalent terms referring to a composition of matter that is suitable for pharmaceutical application. The term “component,” as used herein, refers to an ingredient that may be combined with other components/ingredients so as to produce a composition. In some embodiments, the component or ingredient may be a nutraceutical. In some embodiments, the component or ingredient may be a natural product, namely a chemical compound or substance that is produced by way of one or more living organisms. In some embodiments, the component or ingredient may be a plant or animal extract. It should be understood that the components described herein may be obtained by way of natural sources or may be chemically synthesized.

The term “traumatic brain injury,” as used herein, refers to any injury that may occur when a mechanical force causes damage to the brain. The mechanical force may be internal or external. For example, a traumatic brain injury may result when the head suddenly collides with an object, or when an object pierces the skull and enters brain tissue. Alternatively, a traumatic brain injury may be caused by an “impulsive” force transmitted to the head from other parts of the body. Symptoms of a traumatic brain injury can be mild, moderate, or severe, depending on the extent of the damage to the brain.

As used herein, the term “concussion” refers to a traumatic brain injury that is characterized by an immediate and transient alteration in brain function, including alteration of mental status and level of consciousness. A concussion may be caused by a direct or indirect mechanism, for example a direct blow to the head, face or neck, or a blow elsewhere on the body with an impulsive force transmitted to the head. Diagnosis of concussion includes one or more of the following clinical domains. Symptoms including, but not necessarily limited to, (a) somatic (e.g. headache), cognitive (e.g. feeling like in a fog, dullness) and/or emotional symptoms (e.g. lability, depression), (b) physical signs (e.g. loss of consciousness, amnesia, convulsions), (c) behavioral changes (e.g. irritability), (d) cognitive impairment (e.g. slowed reaction times), (e) sleep disturbance (e.g. drowsiness). Sequelae of concussion may include any of recurrent concussion, migraine headaches, depression, chronic traumatic encephalopathy (CTE), Parkinson's disease, Alzheimer's disease, attention deficit hyperactivity disorder, learning disability, sleep disorders, neurotransmitter production disturbance (e.g. dopamine, serotonin, acetylcholine, GABA) and hormonal disruption/imbalances (e.g. growth hormone, testosterone, pregnenolone, progesterone, estrogen, cortisol).

The term “effective amount,” as used herein, refers to an amount of a particular ingredient that is sufficient to achieve a desired result. As such, once a desired effect is known, determining the effective amount is within the skill of a person skilled in the art. For example, as used herein, an “effective amount of the composition” is optionally a quantity of the composition that is sufficient to treat a subject who has suffered a traumatic brain injury.

The term “pharmaceutically acceptable” is intended to mean compatible with the treatment of animals, and in particular, humans. The terms “treating” or “treatment,” as used herein, and as are well understood in the art, refer to an approach for obtaining beneficial or desired results, including clinical results. Beneficial or desired clinical results may include, but are not limited to, alleviation or amelioration of one or more symptoms or conditions, diminishment of an extent of injury or disease, stabilizing (i.e., not worsening) a state of an injury or disease, delaying or slowing of an injury or disease progression, amelioration or palliation of the injury or disease state, diminishment of a reoccurrence of an injury or disease, and remission (whether partial or total), whether detectable or undetectable.

Treatment methods may comprise administering to a subject a therapeutically effective amount of a composition and may be comprised of a single administration, or a series of applications. The length of a treatment period generally depends on a variety of factors, such as the severity of a condition, the age of a patient, the concentration of the composition and components of the composition, the activity of the compositions and components of the composition, and/or a combination thereof. It will also be appreciated that an effective dosage of the composition and components of the composition used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions may be administered to the subject in an amount and for a duration sufficient to treat the patient.

The term “subject,” as used herein, includes all members of the animal kingdom, including mammals, and particularly refers to humans.

In general, the present disclosure describes compositions comprising a number of individual components. In some embodiments, the components are natural products or nutraceuticals. The compositions are particularly useful for mitigating the effects of traumatic brain injuries, such as concussions. It is contemplated that the compounds described below act synergistically to protect the brain from oxidative damage following a trauma, can stimulate repair of nerve cells due to trauma, reduce inflammation, improve cellular energy production, regenerate neurological capabilities, lessen and correct brain damage associated with head trauma, promote circulation and capillary integrity, limit oxidative stress and augment healing of damaged cervical musculature and connective tissue, stimulate neurogenesis, upregulate the production of new neurons in adult brains, and enhance neural plasticity and repair.

In one embodiment, the composition comprises at least one component from a group that includes Docosahexaenoic acid (DHA), curcumin and resveratrol. In one embodiment, the composition includes linoleic acid (n-6), or α-linoleic acid (n-3). DHA is a major structural component of human brain, cerebral cortex and retina cells, among others. DHA may be derived from natural sources, such as milk, cold-water oceanic fish oils, sunflower oils, soybean oils, or chemically synthesized. In some embodiments, a portion of the composition is comprised of DHA in an amount ranging between substantially 100 mg and 11500 mg, or 1000 mg and 8000 mg, or between 2100 mg and 5500 mg, or substantially 4000 mg.

Curcumin is a principal curcuminoid of spice turmeric. Curcumin may act as an anti-inflammatory agent, increase aerobic capacity and modulate numerous cellular signaling pathways such as inflammatory cytokine production and apoptotic proteins in the present compositions. In some embodiments, the composition is comprised of curcumin in an amount of substantially 100-3000 mg, 200-1500 mg, 400-600 mg, substantially 500, or substantially 800 mg.

Resveratrol is a stilbenoid, a type of natural phenol, and a phytoalexin produced naturally by several plants in response to injury. Resveratrol may be derived from a natural source, such as the skin of grapes, blueberries, raspberries, mulberries, and senna, or chemically synthesized. In some embodiments, the composition is comprised of resveratrol in an amount ranging between substantially 100-3000 mg, 200-1500 mg, 400-600 mg, substantially 500, or substantially 800 mg.

α-linoleic acid (n-3) is an n-3 fatty acid that is one of two essential fatty acids (the other being linoleic acid), so called because they are necessary for health, and they cannot be produced within the human body. α-linoleic acid may be derived from a natural source, such as seeds (chia, flaxseed), nuts (walnuts), and vegetable oils. In some embodiments, the composition is comprised of α-linoleic acid in an amount of 100-3000 mg, 200-1500 mg, 400-600 mg, or substantially 500 mg.

In some embodiments, the composition of the present disclosure may be comprised of any of coenzyme Q10, vitamin K1, vitamin K2, magnesium, potassium, zinc, L-theanine, vitamin B1, vitamin B2, vitamin B3, vitamin B5, vitamin B6, vitamin B9, vitamin B12, vitamin C, vitamin E, valine, leucine, and isoleucine, as well as mixtures thereof.

In some embodiments, the composition comprises 50-200 mg coenzyme Q10, 20-80 mg vitamin K1, 20-80 mg vitamin K2, 200-1000 mg magnesium, 25-150 mg potassium, 5-50 mg zinc, 10-2000 mg L-theanine, 10-50 mg vitamin B complex (B1, 2, 5, 6, 9, 12), 500-2000 mg vitamin C, 50-500 mg vitamin E, and mixtures thereof.

In some embodiments, the composition may be formulated for administration to a subject such as a human. Preferably, the composition is formulated for oral administration. In some embodiments, however, the composition may be formulated for inhalative, rectal or parenteral administration, including dermal, intradermal, intragastral, intracutaneous, intravasal, intravenous, intramuscular, intraperitoneal, intranasal, intravaginal, intrabuccal, percutaneous, subcutaneous, sublingual, and topical or transdermal administration.

The compositions for oral administration may include, but are not limited to, solid, semi-solid, gel, paste, liquid, crystalline, or encapsulated forms. Non-limiting examples of these forms include capsules, tablets, suspensions, powders, suspended-release formulations, solutions, emulsions and syrups. In some embodiments, the composition may be used as an inhalant or suppository. In some embodiments, the compositions for oral administration range from 5 to 50,000 g, 10 to 1000 g, or 15 to 250 g.

In some embodiments, the composition may be formulated such that a single dose is contained in one capsule, tablet, or gel pack. In other embodiments, the composition may be formulated such that a single dose is contained in at least 2, 3, 4 or more individual capsules, tablets, packet, or packets.

In some embodiments, the composition may include a pharmaceutically acceptable carrier, excipient, buffer or stabilizer. Suitable pharmaceutically acceptable carriers include essentially chemically inert and nontoxic materials that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesylphosphotidyl-ethanolamine (DOPE), and liposomes. Such compositions contain a therapeutically effective amount of the components in the composition, together with a suitable amount of carrier so as to provide a form suitable for direct administration to the patient. Further, the composition described herein may include flavor and/or color additives. In some embodiments, for example, beet powder may be added for flavor.

In some embodiments, methods for mitigating a traumatic brain injury comprise administering an effective amount of the above-described compositions to a subject prior to a period wherein a traumatic head injury may be sustained. Further, in some embodiments, an effective amount of the compositions described herein may be administered to the subject during or after the period wherein a traumatic head injury has been sustained.

In one embodiment, the traumatic brain injury may be a concussion. Accordingly, a method for mitigating damage sustained by the concussion comprise administering an effective amount of the compositions described herein to the subject prior to the period wherein the concussion may be sustained. Further, the method may include administering the composition to the subject after suffering a concussion.

In one embodiment, the composition may be administered prior to the onset of any concussion symptoms. In another embodiment, the composition may be used on a subject exhibiting at least 1-5, inclusive, concussion symptoms. Concussion symptoms include, but are not necessarily limited to, headache, pressure in head, neck pain, nausea or vomiting, dizziness, blurred vision, sensitivity to light, sensitivity to noise, feeling slowed down, feeling “in a fog”, “not feeling right”, difficulty concentrating, difficulty remembering, fatigue or low energy, confusion, drowsiness, trouble falling asleep, increased emotions, irritability, sadness and nervousness or anxiety. In some embodiments, the composition may be used on a subject who has been diagnosed with existing traumatic brain injury or a concussion.

In some embodiments, the composition may be administered for preventing or mitigating post-concussive syndrome. Post-concussive syndromes include, but are not necessarily limited to, post-concussion disease, prolonged post-concussion disease, mild cognitive impairment, chronic traumatic encephalopathy and dementia pugilistica. Further, in some embodiments, the composition may be used for preventing or mitigating long-term complications of concussion, such as Alzheimer's disease, Parkinson's disease, amyotrophic lateral sclerosis (ALS) or post concussive depression.

In one embodiment, the composition is administered to the subject one hour prior to a period wherein a traumatic head injury may be sustained. In other embodiments, the composition may be administered between 1 hour and 24 hours, inclusive, prior to a period wherein a traumatic head injury may be sustained. In still other embodiments, the composition may be administered 1, 2, 3, or more days prior to the period wherein a traumatic head injury may be sustained. In other embodiments, the composition may be administered every other day, every third day, once a week, or a combination thereof, prior to the period wherein a traumatic head injury may be sustained.

In one embodiment, the composition may be administered twice a day. In some embodiments, the composition may be administered three times a day, four times a day, or more frequently. In some embodiments, the composition may be administered at least once a day for at least between 1 week and 12 weeks, inclusive, prior to the period wherein a traumatic head injury may be sustained. In some embodiments, the composition may be administered at least once a day for a longer term, such as at least 4, 6, 8, 10, 12 or 24 months prior to the period wherein a traumatic head injury may be sustained. Administration in one embodiment may include, but is not limited to, a dosage of 10-50 mg of the composition at a minimum frequency of 1, 2, 3 or 4 times per day.

In one embodiment, administration continues before, during, and after the period wherein a traumatic head injury has been sustained until all symptoms are resolved and cleared by medical personnel via standardized testing, such as SCAT 2. In some embodiments, the composition may be administered within 1, 2, 3, 5 or 7 days of the traumatic brain injury. In other embodiments, the composition may be administered within 1, 2, 3, 5 or 7 days of the appearance of symptoms of a traumatic brain injury. In one embodiment, the composition may be administered at least once a day until the condition has ameliorated to wherein further treatment is not necessary. In another embodiment, the composition may be administered until all symptoms of the traumatic brain injury are resolved. In another embodiment, the composition may be administered until the subject is able to return to physical activity, or is “cleared to play” a particular sport. In other embodiments, the composition may be administered for at least 1, 2, 3, 6, 8, 10 or 12 or 24 months after the subject is asymptomatic. Further, in some embodiments, the composition may be administered for at least 1, 2, 3, 6, 8, 10 or 12 or 24 months after the subject is able to return to physical activity, or is “cleared to play” a particular sport.

The compositions described herein are useful and effective when administered prior to the period wherein a traumatic head injury, such as a concussion, may be sustained. The amount of each component comprising the composition will be the amount that is determined to be therapeutically effective upon being administered after such an event. Although the therapeutically effective amount will vary depending on the subject and the severity and nature of a given injury, it should be understood that the therapeutically effective amount may be routinely determined based on the individual subject and the nature of the activity undertaken within the period of time wherein a traumatic head injury may occur.

In one embodiment, 100-11500 mg DHA, 100-3000 mg curcumin, and 100-3000 mg of resveratrol is administered to the subject prior to the period wherein a traumatic head injury may be sustained so as to mitigate the effects of a traumatic brain injury such as a concussion. Optionally, 100-3000 mg of α-linoleic acid (n-3) may be administered to the subject prior to the period wherein a traumatic head injury may be sustained so as to mitigate the effects of a traumatic brain injury such as a concussion.

It should be understood that an effective dosage of the composition, as well as the individual components of the composition, suitable for the treatment may increase or decrease over the course of a particular treatment regime. In some embodiments, chronic administration of the composition may be required.

In some embodiments, the composition may be used for treating subjects who are at risk of a traumatic brain injury or who have previously suffered from a traumatic brain injury. In some embodiments, the composition may be administered to the subjects in an effective amount after an onset of symptom of a traumatic brain injury. In other embodiments, the compositions may be used for treating a subject who is suspected of having a traumatic brain injury or a subject who may have suffered from a traumatic brain injury. The subject may or may not display symptoms of a traumatic brain injury.

As mentioned above, it should be understood that the composition described herein is not to be construed as limited solely to mitigating traumatic brain injuries, but rather the composition may be used prior to a variety of conditions that degrade brain and/or central nervous function with which a subject may be predisposed, or may be exhibiting precursor indicators for, or early stage symptoms thereof. Such conditions may include, by way of non-limiting example, Alzheimer's disease, Amyotrophic lateral sclerosis, Friedreich's ataxia, Huntington's disease, Lewy body disease, Parkinson's disease, Spinal muscular atrophy, and the like.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. To the extent there are variations of the invention, which are within the spirit of the disclosure or equivalent to the inventions found in the claims, it is the intent that this patent will cover those variations as well. Therefore, the present disclosure is to be understood as not limited by the specific embodiments described herein, but only by scope of the appended claims.

1. A composition for oral administration prior to a period wherein a traumatic brain injury is expected to occur, comprising;
100-11500 mg of Docosahexaenoic acid;
100-3000 mg of curcumin; and
100-3000 mg of resveratrol.

2. The composition of claim 1, wherein the composition comprises 400 mg of Docosahexaenoic acid, 500 mg of curcumin and 500 mg of resveratrol.

3. The composition of claim 1, wherein the composition further comprises α-linoleic acid.

4. The composition of claim 3, wherein the composition comprises 480 mg of α-linoleic acid.

5. The composition of claim 4, wherein the composition further comprises any one or more of water, saline solutions, glycerol solutions, ethanol, N-(1(2,3-dioleyloxy)propyl)N,N,N-trimethylanrmonium chloride (DOTMA), diolesylphosphotidyl-ethanolamine (DOPE), and liposomes coenzyme Q10, vitamin K1, vitamin K2, magnesium, potassium, zinc, L-theanine, vitamin B1, vitamin B2, vitamin B3, vitamin B5, vitamin B6, vitamin B9, vitamin B12, vitamin C, vitamin E, valine, leucine, or isoleucine.

6. The composition of claim 5, wherein the composition is administered in a gel form.

7. The composition of claim 5, wherein the composition is administered in a slurry form.

8. The composition of claim 5, wherein the composition is administered between one hour and twenty-four hours prior to a period wherein a traumatic brain injury is expected to occur.

9. A method for mitigating the effects of traumatic brain injury in a subject including administering an effective amount of a composition prior to a period wherein the traumatic brain injury is expected to occur, wherein the composition comprises 100-11500 mg Docosahexaenoic acid, 100-3000 mg curcumin and 100-3000 mg resveratrol.

10. The method of claim 9, wherein the composition further includes α-linoleic acid.

11. (canceled)

12. The method of claim 9, wherein the composition further comprises 100-3000 mg of α-linoleic acid.

13. The method of claim 12, wherein the traumatic brain injury is a concussion.

14. The method of claim 12, wherein the composition is administered one to three hours prior to a period wherein the traumatic brain injury is expected to occur.

15. The method of claim 12, wherein the composition is administered three to twenty-four hours prior to the period wherein the traumatic brain injury is expected to occur.

16. The method of claim 12, wherein the composition is administered orally to the subject.

17. The method of claim 12, wherein the composition comprises part of a gel or slurry that is easily absorbed.

18. The method of claim 12, wherein the composition is used prior to an onset of concussion symptoms.

19. (canceled)

20. The method of claim 12, wherein the composition is administered to the subject during or after an onset of concussion symptoms.

21. A method for mitigating the effects of traumatic brain injury in a subject, comprising:
administering a dosage of 10-50 mg of a composition to a subject before an anticipated brain injury or after a traumatic brain injury at a frequency of 1-4 times per day,
wherein the composition comprises 100-11500 mg docosahexaenoic acid, 100-3000 mg curcumin, 100-3000 mg resveratrol, and 100-3000 mg of α-linoleic acid.

22. The method of claim 21, wherein the composition comprises 400 mg docosahexaenoic acid, 500 mg curcumin, 500 mg resveratrol, and 480 mg α-linoleic acid.
